(19) United States
(12) Patent Application Publication
(10) Pub. No.: US 2004/0267010 A1
(43) Pub. Date: Dec. 30, 2004

(54) Sulfonamide compounds, their preparation and use

(76) Inventors: Ian Thomson Forbes, Harlow ,Essex (GB); Andrew Derrik Gribble, Harlow, Essex (GB)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/466,922

(22) PCT Filed: Jan. 2, 2002
(86) PCT No.: PCT/GB02/00447

(30) Foreign Application Priority Data
Feb. 2, 2001 (GB) 0102713.5
Feb. 2, 2001 (GB) 0102714.3

(51) Int. Cl. C07D 417/14; C07D 413/14; C 07D 4 3/14; C 07D 4 1/14

(52) U.S. Cl. 544/49; 544/105; 544/353; 544/284; 546/146; 546/187

(57) ABSTRACT

Compounds possessing 5HT7 activity of formula (I) or a pharmaceutically acceptable salt thereof are disclosed:

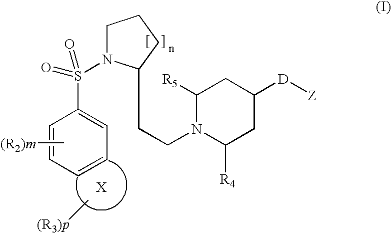

in which X is a 5 or 6 membered heterocyclic ring containing 1 to 3 heteroatoms selected from oxygen, nitrogen and sulfur; R₂ is halogen, C₁₋₆alkyl, haloC₁₋₆alkyl, hydroxy, C₁₋₆alkoxy or C₁₋₆alkylthio; R₃ is hydrogen, C₁₋₆alkyl, hydroxy or oxo; m is 0, 1 or 2; p is 0, 1 or 2; n is 1 or 2; R₄ and R₅ are both hydrogen, or R₄ and R₅ combine together to form a further group:

—(CH₂)q- where q is 2 or 3; D is a single bond, C₁₋₄alkylene, C═O or oxygen; Z is either a group (a):

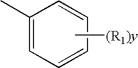

in which R₁ is halogen, C₁-₆alkyl, cyano, haloC₁₋₆alkyl, C₃-₇cycloalkyl, C₁-₆alkoxy, hydroxy, amino, mono- or di-C₁-₆alkylamino, acylamino, nitro, carboxy, C₁-₆alkoxycarbonyl, C₁-₆alkylthio, C₁-₆alkylsulfinyl, C₁-₆alkylsulfonyl, sulfamoyl, mono- and di-C₁-₆alkylsulfamoyl, carbamoyl, mono- or di-C₁-₆alkylcarbamoyl, C₁-₆alkylsulfonamido, arylsulfonamido, aryl, arylC₁-₆alkyl, arylC₁-₆alkoxy, aryloxy or arylthio, and y is 0, 1, 2 or 3;

or a group (b):

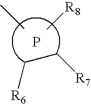

in which P is a 5 membered heterocyclic ring containing 1 to 3 heteroatoms selected from oxygen, nitrogen and sulfur; R₈ is hydrogen, C₁₋₆alkyl, hydroxy or oxo; R₆ and R₇ form the residue of a phenyl ring or a 6 membered heteroaryl ring comprising from one to three heteroatoms selected from nitrogen, oxygen and sulfur and optionally substituted with one or two substitutents which may be the same or different and selected from the group consisting of halogen, C₁-₆alkyl, cyano, haloC₁₋₆alkyl, C₃-₇cycloalkyl, C₁-₆alkoxy, hydroxy, amino, mono- or di-C₁-₆alkylamino, acylamino, nitro, carboxy, C₁-₆alkoxycarbonyl, C₁-₆alkylthio, C₁-₆alkylsulfinyl, C₁-₆alkylsulfonyl, sulfamoyl, mono- and di-C₁-₆alkylsulfamoyl, carbamoyl, mono- and di-C₁-₆alkylcarbamoyl, C₁-₆alkylsulfonamido, arylsulfonamido, aryl, arylC₁-₆alkyl, arylC₁-₆alkoxy, aryloxy and arylthio. Also disclosed are processes for preparation of the compounds, and uses in the treatment of CNS and other disorders.

This invention relates to novel sulfonamide compounds having pharmacological activity, processes for their preparation, to compositions containing them and to their use in the treatment of CNS and other disorders.

WO 97/48681 discloses a series of sulfonamide compounds that are described as possessing 5-HT₇ receptor antagonists and which are claimed to be useful in the treatment of various disorders.

A structurally novel class of compounds have now been found that also possess 5-HT₇ receptor activity. The invention therefore provides, in a first aspect, a compound of formula (I) or a pharmaceutically acceptable salt thereof:

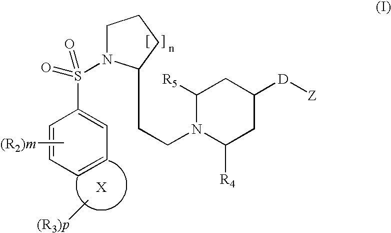

in which

X is a 5 or 6 membered heterocyclic ring containing 1 to 3 heteroatoms selected from oxygen, nitrogen and sulfur;

R₂ Is halogen, C₁₋₆alkyl, haloC₁₋₆alkyl, hydroxy, C₁₋₆alkoxy or C₁₋₆alkylthio;

R₃ is hydrogen, C₁₋₆alkyl, hydroxy or oxo;

m is 0, 1 or2;

p is 0, 1 or 2;

n is 1 or 2;

R₄ and R₅ are both hydrogen or R₄ and R₅ combine together to form a further group

—(CH₂)q- where q is 2 or 3;

D is a single bond, C₁₋₄alkylene, C═O or oxygen;

Z is either

a group (a):

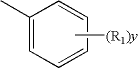

in which R₁ is halogen, C₁₋₆alkyl, cyano, haloC₁₋₆alkyl, C₃₋₇cycloalkyl, C₁₋₆alkoxy, hydroxy, amino, mono- or di-C₁₋₆alkylamino, acylamino, nitro, carboxy, C₁₋₆alkoxycarbonyl, C₁₋₆alkylthio, C₁₋₆alkylsulfinyl, C₁₋₆alkylsulfonyl, sulfamoyl, mono- and di-C₁₋₆alkylsulfamoyl, carbamoyl, mono- or di-C₁₋₆alkylcarbamoyl, C₁₋₆alkylsulfonamido, arylsulfonamido, aryl, arylC₁₋₆alkyl, arylC₁₋₆alkoxy, aryloxy or arylthio, and y is 0, 1, 2 or 3;

or a group (b):

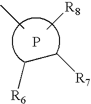

in which P is a 5 membered heterocyclic ring containing 1 to 3 heteroatoms selected from oxygen, nitrogen and sulfur; R₈ is hydrogen, C₁₋₆alkyl, hydroxy or oxo; R₆ and R₇ form the residue of a phenyl ring or a 6 membered heteroaryl ring comprising from one to three heteroatoms selected from nitrogen, oxygen and sulfur and optionally substituted with one or two substitutents which may be the same or different and selected from the group consisting of halogen, C₁₋₆alkyl, cyano, haloC₁₋₆alkyl, C₃₋₇cycloalkyl, C₁₋₆alkoxy, hydroxy, amino, mono- or di-C₁₋₆alkylamino, acylamino, nitro, carboxy, C₁₋₆alkoxycarbonyl, C₁₋₆alkylthio, C₁₋₆alkylsulfinyl, C₁₋₆alkylsulfonyl, sulfamoyl, mono- and di-C₁₋₆alkylsulfamoyl, carbamoyl, mono- and di-C₁₋₆alkylcarbamoyl, C₁₋₆alkylsulfonamido, arylsulfonamido, aryl, arylC₁₋₆alkyl, arylC₁₋₆alkoxy, aryloxy and arylthio.

The term “heterocyclic ring containing 1 to 3 heteroatoms selected from oxygen, nitrogen and sulfur” refers to saturated and non-saturated heterocyclic rings. Examples of 5 membered heterocyclic rings include pyrrolyl, pyrrolinyl, pyrazolinyl, imidazolyl, thiazolyl, furyl, thienyl, pyrrolidinyl, imidazolidinyl, oxazolidinyl, thiazolidinyl, tetrahydrofuryl, tetrahydrothienyl and diaoxolanyl. Examples of 6 membered heterocyclic rings include pyridyl, pyrimidinyl, pyrazinyl, piperidyl, piperazinyl, morpholinyl and thiomorpholinyl.

The term “halogen” is used herein to describe, unless otherwise stated, fluorine, chlorine, bromine or iodine.

The term “aryl” whether alone or as part of another group is used herein to describe, unless otherwise stated, an aromatic carbocyclic or heterocyclic group such as phenyl, naphthyl, pyridyl or pyrazinyl. Such aryl groups may be optionally substituted by one or more C₁₋₆alkyl or halogen.

The term “alkyl”, whether alone or as part of another group, is used herein to describe a straight chain or branched fully saturated hydrocarbon group. “C₁₋₆alkyl” refers to alkyl groups having from one to six carbon atoms, including all isomeric forms, such as methyl, ethyl, propyl, isopropyl, butyl, isobutyl, sec-butyl, tert-butyl, pentyl, neopentyl, sec-pentyl, n-pentyl, isopentyl, tert-pentyl and hexyl.

The term “haloC₁₋₆alkyl” is used herein to describe a C₁₋₆alkyl group substituted by one or more halogen atoms, such as CF₃.

The term “C₁₋₆alkoxy” refers to a straight chain or branched chain alkoxy (or alkyloxzy) group having from one to six carbon atoms, including all isomeric forms, such as methoxy, ethoxy, propoxy, isopropoxy, butoxy, isobutoxy, sec-butoxy, tert-butoxy, pentoxy, neopentoxy, sec-pentoxy, n-pentoxy, isopentoxy, tert-pentoxy and hexoxy.

The term “C₁₋₆alkylthio” refers to a straight chain or branched chain alkylthio group having from one to six carbon atoms, such as methylthio, ethylthio, propylthio, isopropylthio, butylthio, isobutylthio, sec-butylthio, tert-butylthio, pentylthio, neopentylthio, sec-pentylthio, n-pentylthio, isopentylthio, tert-pentylthio and hexylthio.

The term “oxo” refers to the group “═O”.

The term “C₁₋₄alkylene” refers to methylene, ethylene, propylene or butylene.

The term “C₃₋₇cycloalkyl” refers to cycloalkyl groups consisting of from 3 to 7 carbon atoms, such as cyclopropane, cyclobutane, cyclopentane, cyclohexane or cycloheptane.

The term “mono- or di-C₁₋₆alkylamino” refers to an amino group which is substituted by one C₁₋₆alkyl group or an amino group which is substituted by two C₁₋₆alkyl groups, the two amino groups being the same or different. Examples of monoC1-6alkylamino include methylamine, ethylamine, propylamine, isopropylamine, butylamine, isobutylamine, sec-butylamine, tert-butylamine, pentylamine, neopentylamine, sec-pentylamine, n-pentylamine, isopentylamine, tert-pentylamine and hexylamine. Examples of di-C1-6alkylamino include dimethylamine, diethylamine, dipropylamine, diisopropylamine, dibutylamine, diisobutylamine, disec-butylamine, ditert-butylamine, dipentylamine, dineopentylamine, dihexylamine, butylmethylamino, isopropylmethylamino, ethylisopropylamino, ethylmethylamino, etc.

The term “6 membered heteroaryl ring containing 1 to 3 heteroatoms selected from oxygen, nitrogen and sulfur” refers to 6 membered unsaturated heterocyclic rings such as pyridyl, pyridazinyl, pyrimidinyl or pyrazinyl.

Preferably, the 5 or 6 membered heterocyclic ring represented by X contains at least one nitrogen, most preferably containing a free NH group with a meta relationship with respect to the sulfonamide linkage. Preferably the heterocyclic ring X, together with the benzene ring to which it is fused, forms an indole, indoline, indazole, benzotriazole, benzimidazole or a benzoxazine group. Most preferably, the heterocyclic ring X, together with the benzene ring to which it is fused, forms an indole, indazole or benzoxazinone group.

When m is 2, the two R₂ groups can be the same or different. Preferred examples of R₂ groups are C₁₋₆alkyl (particularly methyl), halogen (particularly fluoro or chloro), C₁₋₆alkoxy (particularly methoxy) or haloC₁₋₆alkyl (particularly CF₃). Preferably m is 0.

When p is 2, the two R₃ groups can be the same or different. When R₃ is C₁₋₆alkyl, preferred groups include methyl and ethyl. Such groups may be substituted on any suitable carbon or nitrogen atom. It will be appreciated that when R₃ is hydroxy or oxo, compounds may exist In more than one tautomeric form.

Preferably n is 1. When n is 1, the preferred stereochemistry of the pyrrolidine ring is R.

When R₄ and R₅ combine together to form a group —(CH₂)q-, q is preferably 2. Preferably R₄ and R₅ are both hydrogen.

When D is a C₁₋₄alkylene group, preferred examples are methylene and ethylene.

Preferred compounds are compounds of formulae (Ia):

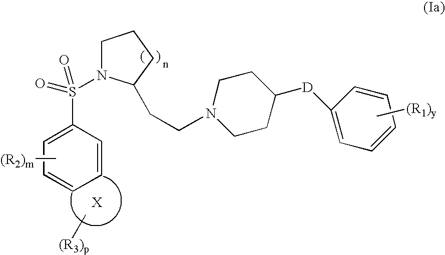

wherein X, R₁, R₂, R₃, m, p, y and n have the same meanings as defined for general formula (1), and D is C═O or oxygen;

and formula (Ib):

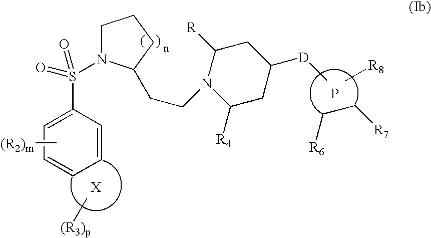

wherein X, R₂, R₃ R_{4,} R₅, R₆ R₇, R₈, m, p, q, P and n have the same meanings as defined for general formula (I), and D is a single bond or a C₁₋₄alkylene group.

In formula (Ib) above, D is preferably a single bond.

In formula (Ia) above, when y is one or more, R₁ is preferably C₁₋₆alkyl (particularly methyl), halogen (particularly fluoro or chloro), C₁₋₆alkoxy (particularly methoxy) or haloC₁₋₆alkyl (particularly CF₃). When y is two or more, the two or more R₁ groups can be the same or different. Preferably y is 0, 1 or 2. A particularly preferred R₁ group is halogen, most preferably 4-fluoro or 4-chloro.

In formula (Ib) above, suitably P is a 5 membered heterocyclic ring such as pyrrolyl, thienyl, furyl, Imidazolyl, oxazolyl or thiazolyl. Such groups can be linked to the remainder of the molecule via a carbon atom or, when present, a suitable nitrogen atom. Preferably R₈ is hydrogen or oxo. Preferably, R₆ and R₇ form a phenyl ring or R₆ and R₇ form a 6 membered heteroaryl ring comprising one nitrogen atom, such as a pyridyl ring. Preferred substituents for the phenyl ring or the heteroaryl ring formed by R₆ and R₇ include C₁₋₆alkyl (particularly methyl or ethyl), halogen (particularly fluoro and chloro), C₁₋₆alkoxy (particularly methoxy) and haloC₁₋₆alkyl (particularly CF₃).

Other preferred features of general formula (I) apply to general formulae (Ia) and (Ib) mutatis mutandis.

Particularly preferred compounds of this invention include Examples 1-29 as shown below or a pharmaceutically acceptable salt thereof.

The compounds of formula (I) can form acid addition salts thereof. It will be appreciated that for use in medicine the salts of the compounds of formula (I) should be pharmaceutically acceptable. Suitable pharmaceutically acceptable salts will be apparent to those skilled in the art and include those described in J. Pharm. Sci., 1977, 66, 1-19, such as acid addition salts formed with inorganic acids e.g. hydrochloric, hydrobromic, sulfuric, nitric or phosphoric acid; and organic acids e.g. succinic, maleic, acetic, fumaric, citric, tartaric, benzoic, p-toluenesulfonic, methanesulfonic or naphthalenesulfonic acid.

The compounds of formula (I) may be prepared in crystalline or non-crystalline form, and, if crystalline, may optionally be hydrated or solvated. This invention includes within its scope stoichiometric hydrates as well as compounds containing variable amounts of water.

Certain compounds of formula (I) are capable of existing in stereoisomeric forms (e.g. geometric or (“cis-trans”) isomers, diastereomers and enantiomers) and the invention extends to each of these stereoisomeric forms and to mixtures thereof including racemates. The different stereoisomeric forms may be separated one from the other by the usual methods, or any given isomer may be obtained by stereospecific or asymmetric synthesis. The invention also extends to any tautomeric forms and mixtures thereof.

The present invention also provides, in a further aspect, a process for the preparation of a compound of formula (I) or a pharmaceutically acceptable salt thereof, which process comprises either

(a) coupling a compound of formula (II):

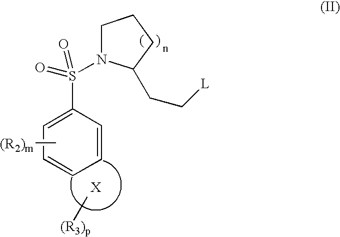

in which X, R₂, R₃, m, n and p are as defined for formula (I) and L is a leaving group, e.g. halogen, with a compound of formula (III):

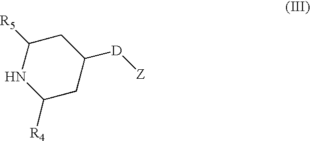

in which Z, R₁, y, R₆, R₇, R₈, R₄, R₅ and D are as defined for formula (I); or

(b) formation from a compound of formula (IV)

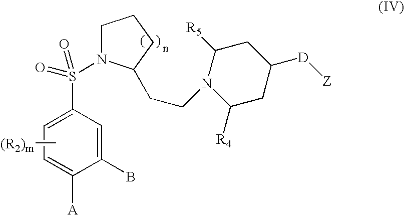

in which R₂, R₄, R₅, q, D, Z, R₁, y, R₆, R₇, R₈, n and m are as defined for formula (I) and A and B are appropriate functional groups for the formation of the ring X;

and optionally thereafter if appropriate for either (a) or (b):

converting a compound of formula (I) into a further compound of formula (I)

removing any protecting groups;

forming pharmaceutically acceptable salt.

For process (a) the reaction of compounds of formulae (II) and (III) are preferably carried out in a solvent such as dichloromethane or acetonitrile optionally in the presence of a base such as triethylamine. Preferably L is chloro.

For process (b) suitable functional groups include those known to the person skilled in the art. By way of illustration, suitable examples of A and B groups include A=Me and B=NO₂ (Leimgruber synthesis—Comp. Het. Chem., Vol, 4, page 328) and A=NH₂ and Z=NO₂ involving reduction of the nitro group and reaction with phosgene or a phosgene equivalent (Comp. Het. Chem., vol. 5, p. 471).

Compounds of formula (I) can be converted into further compounds of formula (I) using standard techniques. By way of illustration rather than limitation, compounds of formula (I) wherein X, together with the benzene ring to which it is fused, forms an indole ring such compounds can be converted into the corresponding indoline derivatives by reduction with a reducing agent for example, sodium cyanoborohydride or pyridine-borane complex.

It will be appreciated by those skilled in the art that it may be necessary to protect certain reactive substituents during some of the above procedures. Standard protection and deprotection techniques, such as those described in Greene T. W. ‘Protective groups in organic synthesis’, New York, Wiley (1981), can be used. For example, primary amines can be protected as phthalimide, benzyl, benzyloxycarbonyl or trityl derivatives. Carboxylic acid groups can be protected as esters. Aldehyde or ketone groups can be protected as acetals, ketals, thioacetals or thioketals. Deprotection of such groups is achieved using conventional procedures well known in the art.

Compounds of formulae (II), (III) and (IV) can be prepared by methods described herein or by analogous methods thereto, are commercially available or may be prepared according to known methods or analogous to known methods.

Pharmaceutically acceptable salts may be prepared conventionally by reaction with the appropriate acid or acid derivative.

Compounds of formula (I) and their pharmaceutically acceptable salts have 5-HT₇ receptor antagonist activity and are believed to be of potential use for the treatment of CNS and other disorders such as anxiety disorders, including generalised anxiety; depression including bipolar depression and unipolar depression, single or recurrent major depressive episodes with or without psychotic features, catatonic features, melancholic features, atypical features or postpartum onset, depression resulting from generalised medical condition including, but not limited to, myocardial infarction, diabetes, miscarriage or abortion etc, seasonal affective disorder and dysthymia; panic disorder, agoraphobia; social phobia; obsessive compulsive disorder; schizophrenia; post-traumatic stress disorder, attention deficit disorders; sleep disorders, including disturbances of circadian rhythms, dyssomnia, insomnia, sleep apnea and narcolepsy; migraine; neurodegenerative disorders such as Parkinson's disease and Alzheimers disease; pain disorders including neuropathic pain, diabetic neuropathy, chronic back pain, post-herpetic neuralgia, AIDS-associated neuropathy, neuropathy arising from peripheral nerve injury, complex-regional pain syndrome, trigeminal neuralgia, cancer pain and sciatica; memory disorders including dementia, amnesic disorders and age-associated memory impairment; feeding disorders such as anorexia and bulimia; sexual dysfunction; withdrawal from abuse of drugs such as of cocaine, ethanol, nicotine, benzodiazepines, alcohol, caffeine, phencyclidine (phencyclidine-like compounds), opiates (e.g. cannabis, heroin, morphine), sedative ipnotic, amphetamine or amphetamine-related drugs (e.g. dextroamphetamine, methylamphetamine) or a combination thereof; ocular disorders; asthma; epilepsyl; hypothalamic diseases; inflammation; renal disorders including urinary incontinence; hypotension; cardiovascular shock; stroke including neurodegeneration resulting from stroke; septic shock and gastrointestinal diseases such as spastic colon and IBS (irritable bowel syndrome).

It will be appreciated that reference to treatment is intended to include prophylaxis as well as the alleviation of established symptoms.

Thus the invention also provides a compound of formula (I) or a pharmaceutically acceptable salt thereof, for use as a therapeutic substance, in particular in the treatment of the above disorders. In particular the invention provides a compound of formula (I) or a pharmaceutically acceptable salt thereof for use in the treatment of depression, anxiety, migraine, stroke, pain and/or sleep disorders.

Compounds of the invention may be administered in combination with other active substances such as 5HT3 antagonists, serotonin agonists, NK-1 antagonists, selective serotonin reuptake inhibitors (SSRI), noradrenaline re-uptake inhibitors (SNRI), tricyclic antidepressants and/or dopaminergic antidepressants.

Suitable 5HT3 antagonists which may be used in combination of the compounds of the inventions include for example ondansetron, granisetron, metoclopramide.

Suitable serotonin agonists which may be used in combination with the compounds of the invention include sumatriptan, rauwolscine, yohimbine, metoclopramide.

Suitable SSRIs which may be used in combination with the compounds of the invention include fluoxetine, citalopra, femoxetine, fluvoxamine, paroxetine, indalpine, sertraline, zimeldine.

Suitable SNRIs which may be used in combination with the compounds of the invention include venlafaxine and reboxetine.

Suitable tricyclic antidepressants which may be used in combination with a compound of the invention include imipramine, amitriptiline, chlomipramine and nortriptiline.

Suitable dopaminergic antidepressants which may be used in combination with a compound of the invention include bupropion and amineptine.

It will be appreciated that the compounds of the combination or composition may be administered simultaneously (either in the same or different pharmaceutical formulations), separately or sequentially.

The invention further provides a method of treatment of the above disorders, in mammals including humans, which comprises administering to the sufferer a therapeutically effective amount of a compound of formula (I) or a pharmaceutically acceptable salt thereof.

In another aspect, the invention provides the use of a compound of formula (I) or a pharmaceutically acceptable salt thereof in the manufacture of a medicament for the treatment of the above disorders.

In order to use the compounds of formula (I) in therapy, they will normally be formulated into a pharmaceutical composition in accordance with standard pharmaceutical practice. The present invention also provides a pharmaceutical composition, which comprises a compound of formula (I) or a pharmaceutically acceptable salt thereof and a pharmaceutically acceptable carrier or excipient.

In a further aspect, the present invention provides a process for preparing a pharmaceutical composition, the process comprising mixing a compound of formula (I) or a pharmaceutically acceptable salt thereof and a pharmaceutically acceptable carrier or excipient.

A pharmaceutical composition of the invention, which may be prepared by admixture, suitably at ambient temperature and atmospheric pressure, is usually adapted for oral, parenteral or rectal administration and, as such, may be in the form of tablets, capsules, oral liquid preparations, powders, granules, lozenges, reconstitutable powders, injectable or infusable solutions or suspensions or suppositories. Orally administrable compositions are generally preferred.

Tablets and capsules for oral administration may be in unit dose form, and may contain conventional excipients, such as binding agents (e.g. pregelatinised maize starch, polyvinylpyrrolidone or hydroxypropyl methylcellulose);, fillers (e.g. lactose, microcrystalline cellulose or calcium hydrogen phosphate); tabletting lubricants lubricants (e.g. magnesium stearate, talc or silica); disintegrants (e.g. potato starch or sodium starch glycollate); and acceptable wetting agents (e.g. sodium lauryl sulfate). The tablets may be coated according to methods well known in normal pharmaceutical practice.

Oral liquid preparations may be in the form of, for example, aqueous or oily suspension, solutions, emulsions, syrups or elixirs, or may be in the form of a dry product for reconstitution with water or other suitable vehicle before use. Such liquid preparations may contain conventional additives such as suspending agents (e.g. sorbitol syrup, cellulose derivatives or hydrogenated edible fats), emulsifying agents (e.g. lecithin or acacia), non-aqueous vehicles (which may include edible oils e.g. almond oil, oily esters, ethyl alcohol or fractionated vegetable oils), preservatives (e.g. methyl or propyl-p-hydroxybenzoates or sorbic acid), and, if desired, conventional flavourings or colorants, buffer salts and sweetening agents as appropriate. Preparations for oral administration may be suitably formulated to give controlled release of the active compound.

For parenteral administration, fluid unit dosage forms are prepared utilising a compound of the invention or pharmaceutically acceptable salt thereof and a sterile vehicle. Formulations for injection may be presented in unit dosage form e.g. in ampoules or in multi-dose, utilising a compound of the invention or pharmaceutically acceptable salt thereof and a sterile vehicle, optionally With an added preservative. The compositions may take such forms as suspensions, solutions or emulsions in oily or aqueous vehicles, and may contain formulatory agents such as suspending, stabilising and/or dispersing agents. Alternatively, the active ingredient may be in powder form for constitution with a suitable vehicle, e.g. sterile pyrogen-free water, before use. The compound, depending on the vehicle and concentration used, can be either suspended or dissolved in the vehicle. In preparing solutions, the compound can be dissolved for injection and filter sterilised before filling into a suitable vial or ampoule and sealing. Advantageously, adjuvants such as a local anaesthetic, preservatives and buffering agents are dissolved in the vehicle. To enhance the stability, the composition can be frozen after filling into the vial and the water removed under vacuum. Parenteral suspensions are prepared in substantially the same manner, except that the compound is suspended in the vehicle instead of being dissolved, and sterilisation cannot be accomplished by filtration. The compound can be sterilised by exposure to ethylene-oxide before suspension in a sterile vehicle. Advantageously, a surfactant or wetting agent is included in the composition to facilitate uniform distribution of the compound.

Lotions may be formulated with an aqueous or oily base and will in general also contain one or more emulsifying agents, stabilising agents, dispersing agents, suspending agents, thickening agents, or colouring agents. Drops may be formulated with an aqueous or non-aqueous base also comprising one or more dispersing agents, stabilising agents, solubilising agents or suspending agents. They may also contain a preservative.

The compounds of the invention may also be formulated in rectal compositions such as suppositories or retention enemas, e.g. containing conventional suppository bases such as cocoa butter or other glycerides.

The compounds of the invention may also be formulated as depot preparations. Such long acting formulations may be administered by implantation (for example subcutaneously or intramuscularly) or by Intramuscular injection. Thus, for example, the compounds of the invention may be formulated with suitable polymeric or hydrophobic materials (for example as an emulsion in an acceptable oil) or ion exchange resins, or as sparingly soluble derivatives, for example, as a sparingly soluble salt.

For intranasal administration, the compounds of the invention may be formulated as solutions for administration via a suitable metered or unitary dose device or alternatively as a powder mix with a suitable carrier for administration using a suitable delivery device. Thus compounds of formula (I) may be formulated for oral, buccal, parenteral, topical (including ophthalmic and nasal), depot or rectal administration or in a form suitable for administration by inhalation or insufflation (either through the mouth or nose).

The compounds of the invention may be formulated for topical administration in the form of ointments, creams, gels, lotions, pessaries, aerosols or drops (e.g. eye, ear or nose drops). Ointments and creams may, for example, be formulated with an aqueous or oily base with the addition of suitable thickening and/or gelling agents. Ointments for administration to the eye may be manufactured in a sterile manner using sterilised components.

The composition may contain from 0.1% to 99% by weight, preferably from 10 to 60% by weight, of the active material, depending on the method of administration.

The dose of the compound used in the treatment of the aforementioned disorders will vary in the usual way with the seriousness of the disorders, the weight of the sufferer, and other similar factors. However, as a general guide suitable unit doses may be 0.05 to 1000 mg, more suitably 0.05 to 20.0 mg, for example 0.2 to 5 mg; and such unit doses may be administered more than once a day, for example two or three times a day, so that the total daily dosage is in the range of about 0.5 to 100 mg; and such therapy may extend for a number of weeks or months.

All publications, including but not limited to patents and patent applications, cited in this specification are herein incorporated by reference as if each individual publication were specifically and individually indicated to be incorporated by reference herein as though fully set forth.

The following Descriptions and Examples illustrate the preparation of the compounds of the invention. NMR and mass spectra were consistent with the structures shown.

### DESCRIPTION 1

### 6-[(R)-2-(2-Bromo-ethyl)-pyrrolidine-1-sulfonyl]-1H-indole (D1)

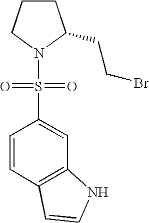

The title compound was prepared using the following steps:—

### a) Preparation of (R)-2-Formyl-pyrrolidine-1-carboxylic acid, t-butyl ester

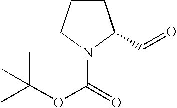

A stirred solution of (+) N-BOC-D-prolinol (11.26 g, 55.9 mmol) in dichloromethane (240 mL) was treated with powdered molecular sieves (16 g) followed by pyridinium dichromate (25.4 g). After stirring for 2 h, the mixture was diluted with ethyl acetate, then filtered through celite. Evaporation of the solvent afforded the crude product which was purified by chromatography on silica, eluting with 25-50% ethyl acetate-hexane gave the pure product (8 g, 72%).

¹H NMR CDCl₃ complex due to presence of rotamers (aldehyde protons at δ9.46 and 9.56)

### b) Preparation of (R)-2-Vinyl-pyrrolidine-1-carboxylic acid, t-butyl ester

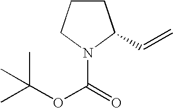

Sodium hydride (60% in oil, 1.65 g) was added to dry dimethylsulfoxide (50 mL) under argon, and the mixture was heated to 60° C. for 1 h. The solution was then cooled to 0° C. and a solution of methyltriphenylphosphonium bromide (14.4 g, 40.3 mmol) in dry dimethylsulfoxide (50 mL) added dropwise. After stirring for 30 min, a solution of (R)-2-formyl-pyrrolidine-1-carboxylic acid t-butyl ester (8.0 g, 40.2 mmol) in dry dimethylsulfoxide (15 mL) was added dropwise. The solution was stirred for 18 h at room temperature, then partitioned between water and 70% ethyl acetate-hexane. The organics were dried and evaporated to dryness. Chromatography on silica, eluting with 10% ethyl acetate-hexane gave the pure product (6.3 g, 79%)

¹H NMR CDCl₃ δ: 1.44 (9H, s), 1.6-2.1 (4H, m), 3.38 (2H, t), 4.26 (1H, b s), 5.04 m), 5.71 (1H, m).

### c) Preparation of (R)-2-(2-Hydroxy-ethyl)-pyrrolidine-1-carboxylic acid, t-butyl ester

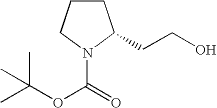

To a solution of (R)-2-vinyl-pyrrolidine-1-carboxylic acid t-butyl ester (6.3 g, 32 mmol) in dry tetrahydrofuran (130 mL) at 0° C. under argon was added 0.5M 9-BBN (192 mL). The solution was then refluxed for 18 h, then cooled. Ethanol (80 mL) was added cautiously followed by 2M sodium hydroxide (100 mL), followed by 30% hydrogen peroxide (60 mL) dropwise with cooling. The mixture was then refluxed for 3 h, then allowed to cool and diluted with brine. The mixture was extracted with ethyl acetate (×3), and the combined organics washed with brine, dried and evaporated to afford the crude product. Chromatography on silica, eluting with 20-40% ethyl acetate-hexane gave the pure product (6.7 g, 98%)

¹H NMR CDCl₃ δ: 1.47 (9H, s), 1.63 (2H, b s), 1.8-2.0 (4H, m), 3.16 (2H, m), 3.59 (2H, m), 4.13 (1H, b s), 4.37 (1H, b s)

### d) Preparation of (R)2-Pyrrolidin-2-yl-ethanol hydrochloride

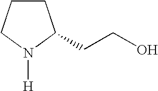

A solution of (R)-2-(2-Hydroxy-ethyl)-pyrrolidine-1-carboxylic acid t-butyl ester (2.5 g, 11.6 mmol) in ethanol (10 mL) was treated with saturated ethanolic hydrogen chloride (30 mL). After stirring for 30 min, the solution was evaporated to dryness to afford the crude product (1.87 g, 100%) which was used directly in the next step.

### e) 2-[(R)-1-(4-Methyl-3-nitro-benzenesulfonyl)-pyrrolidin-2-yl]-ethanol

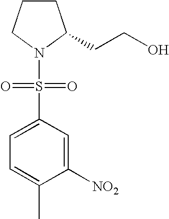

A mixture of (R)-2-pyrrolidin-2-yl-ethanol hydrochloride (5.1 g, 32.5 mmol) in dichloromethane (100 mL) was treated with diisopropylethylamine (15 mL) followed by 4-methyl-3-nitro-benzenesulfonyl chloride (8.4 g, 35.7 mmol) in dichloromethane (75 mL). The mixture was stirred at room temperature for 18 h under argon. The reaction mixture was treated with sodium bicarbonate solution then extracted with dichloromethane. The organic extracts were dried and evaporated to dryness. Chromatography on silica, eluting with 0-2.5% methanol in dichloromethane gave the pure product (10.3 9,-100%). Mass spectrum MH⁺ 315.

### f) (R)-2-[2-(2-Methoxy-ethoxymethoxy)-ethyl]-1-(4-methyl-3-nitro-benzenesulfonyl)-pyrrolidine

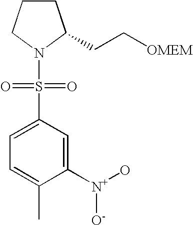

A solution of 2-[(R)-1-(4-methyl-3-nitro-benzenesulfonyl)-pyrrolidin-2-yl]-ethanol (1.0 g, 3.18 mmol) in dichloromethane containing diisopropylethylamine (0.85 mL) was treated with MEM chloride (0.6 g, 4.8 mmol) in dichloromethane (2 mL) at room temperature under argon. After stirring for 18 h, the reaction mixture was poured onto water and extracted with dichloromethane (2×). The organic extracts were combined, dried and evaporated, then chromatographed over silica using 40-70% ethyl acetate/hexane as eluent to afford the title compound (1.13 g, 89%). Mass spectrum MH⁺ 403.

### g) 6-{(R)-2-[2-(2-Methoxy-ethoxymethoxy)-ethyl]-pyrrolidine-1-sulfonyl}-1H-indole

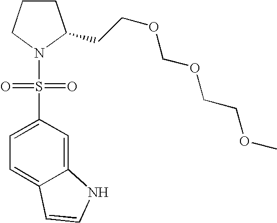

A solution of (R)-2-[2-(2-Methoxy-ethoxymethoxy)-ethyl]-1-(4-methyl-3-nitro-benzenesulfonyl)-pyrrolidine (1.13 g, 2.8 mmol) in dimethylformamide dimethyl acetal (8 mL) containing dimethylformamide (1 mL) was heated to 100° C. for 8 h, then evaporated to dryness. The crude intermediate was then dissolved in tetrahydrofuran (15 mL) and methanol (15 mL), then Raney nickel (1 spoonful) added. Addition of hydrazine hydrate (2 mL) over a 2 h period was carried out, keeping the internal temperature at 45° C. The reaction mixture was then cooled, and filtered, then evaporated to dryness. Chromatography on silica using 40-70% ethyl acetate/hexane as eluent afforded the pure product (0.66 g, 62%). Mass spectrum MH⁺ 383.

### h) 2-[(R)-1-(1H-Indole-6-sulfonyl)-pyrrolidin-2-yl]-ethanol

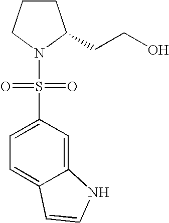

A solution of 6-{(R)-2-[2-(2-methoxy-ethoxymethoxy)-ethyl]-pyrrolidine-1-sulfonyl)-1H-indole (0.6 g, 1.57 mmol) in dimethylformamide (10 mL) was treated with 60% sodium hydride (80 mg, 1.9 mmol) followed by benzenesulfonyl chloride (305 mg, 1.73 mmol). After stirring for 3 h, the reaction mixture was poured onto brine and extracted with ethyl acetate. The organic layers were combined, washed with brine, dried and evaporated to afford the protected indole-N-phenylsulfonamide. This intermediate was dissolved in dioxan (20 mL) and treated with 5M HCl (5 mL), then allowed to stir overnight at room temperature. The reaction mixture was poured onto brine and extracted with ethyl acetate. The organic layer was washed with brine, dried and evaporated to afford the deprotected alcohol. Mass spectrum MH⁺ 435. Removal of the indole-N-phenylsulfonamide protecting group was accomplished by treatment with 10% sodium hydroxide solution (3 mL) in dioxan (10 mL) at room temperature. After stirring for 18 h, the reaction mixture was poured onto water and extracted with ethyl acetate. The organic layer was washed with brine, dried and evaporated. Chromatography of the crude product on silica using 30-80% ethyl acetat/hexane as eluent afforded the pure product (270 mg, 65%). Mass spectrum MH⁺ 295.

### i) 6-[(R)-2-(2-Bromo-ethyl)-pyrrolidine-1-sulfonyl]-1H-indole (title compound)

A solution of 2-[(R)-1-(1H-indole-6-sulfonyl)pyrrolidin-2-yl]-ethanol (190 mg, 0.64 mmol) in dichloromethane (4 mL) was cooled to 0° C. and then treated with carbon tetrabromide (254 mg, 0.77 mmol) followed by triphenylphosphine (200 mg, 0.77 mmol). Chromatography of the reaction mixture directly onto a short column of silica, and elution with dichloromethane afforded the pure product (200 mg, 88%). Mass spectrum MH⁺ 358.

### DESCRIPTION 2

### 6-[(R)-2-(2-Bromo-ethyl)-pyrrolidine-1-sulfonyl]-1H-indazole (D2)

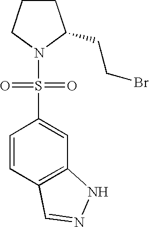

The title compound was prepared from Description 1e using the following steps:—

### a) 2-[(R)-1-(3-Amino-4-methyl-benzenesulfonyl)-pyrrolidin-2-yl]-ethanol

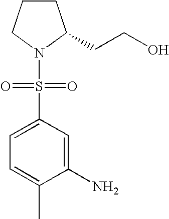

To a solution of 2-[(R)1-(4-methyl-3-nitro-benzenesulfonyl)pyrrolidin-2-yl]-ethanol D1e (8.2 g, 26 mmol) in tetrahydrofuran (80 mL) and methanol (80 mL) was added Raney nickel (1 spoonful). The mixture was then treated with hydrazine hydrate (3×4 mL additions over a 2 h period) keeping the internal temperature at 45° C. After cooling the catalyst was filtered off, and the solution evaporated to dryness to afford the pure product (6.97 g, 95%). Mass spectrum MH⁺ 285.

### b) 2-[(R)-1-(1H-Indazole-6-sulfonyl)-pyrrolidin-2-yl]-ethanol

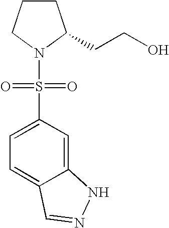

A solution of 2-[(R)-1-(3-amino-4-methyl-benzenesulfonyl)-pyrrolidin-2-yl]-ethanol (3.4 g, 11.9 mmol) in acetic acid (40 mL) and water (10 mL) was cooled in an ice bath, then a solution of sodium nitrite (1.24 g, 18 mmol) in water (3 mL) was added dropwise. The mixture was allowed to warm to room temperature then stirred for 18 h. The reaction mixture was then neutralised with 40% sodium hydroxide (˜65 mL) with cooling, and extracted with ethyl acetate (×2). The combined organic layer was washed with brine, dried and evaporated to afford the crude product. Chromatography on silica with 5080% ethyl acetate in hexane as eluent afforded the pure product (2.5 g, 71%). Mass spectrum MH⁺ 296.

### c) 6-[(R)-2-(2-Bromo-ethyl)-pyrrolidine-1-sulfonyl]-1H-indazole (title compound)

6-[(R)-2-(2-Bromo-ethyl)pyrrolidine-1-sulfonyl]-1H-indazole was prepared in 77% yield from 2-[(R)-1-(1H-indazole-6-sulfonyl)-pyrrolidin-2-yl]-ethanol using a procedure similar to that in Description 1i. Mass spectrum MH⁺ 359.

### DESCRIPTION 3

### 6-[(R)-2-(2-Bromo-ethyl)-pyrrolidine-1-sulfonyl]-4H-benzo[1,4]oxazin-3-one (D3)

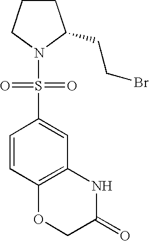

The title compound was prepared using the following steps:—

### a) 6-[(R)-2-(2-Hydroxy-ethyl)-pyrrolidine-1-sulfonyl]-4H-benzo[1,4]oxazin-3-one

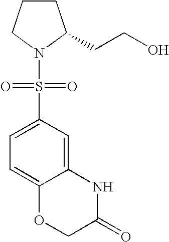

A mixture of (R)-2-pyrrolidin-2-yl-ethanol hydrochloride (3.3 g, 18.6 mmol) in dichloromethane (60 mL) was treated with diisopropylethylamine (9 mL) followedby 3-oxo-3,4-dihydro-2H-benzo[1,4]oxazine-6-sulfonyl chloride (C R Acad Science Ser C. 1970, 270, 1601) (5.2 g, 21 mmol) in dichloromethane (20 mL). The mixture was stirred at room temperature for 18 h under argon. The reaction mixture was treated with sodium bicarbonate solution then extracted with dichloromethane. The organic extracts were dried and evaporated to dryness. Chromatography on silica, eluting with 2-5% methanol in dichloromethane gave the pure product (6.0 g, 98%). Mass spectrum MH⁺ 327.

### b) 6-[(R)-2-(2-Bromo-ethyl)-pyrrolidine-1-sulfonyl]-4H-benzo[1,4]oxazin-3-one (title compound)

6-[(R)-2-(2-Bromo-ethyl)-pyrrolidine-1-sulfonyl]-4H-benzo[1,4]oxazin-3-one was prepared in 86% yield from 6-[(R)-2-(2-hydroxy-ethyl)-pyrrolidine-1-sulfonyl]4H-benzo[1,4]oxazin-3-one using a procedure similar to that in Description 1i. Mass spectrum MH⁺ 390.

### DESCRIPTION 4

### 6-[(R)-2-(2-Bromo-ethyl)-pyrrolidine-1-sulfonyl]-1H-indole (D1)

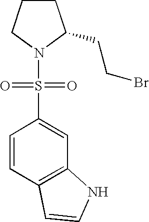

The title compound was prepared using the following steps:—

### a) Preparation of (R)-2-Formyl-pyrrolidine-1-carboxylic acid, t-butyl ester

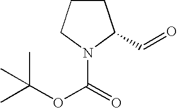

A stirred solution of (+) N-BOC-D-prolinol (11.26 g, 55.9 mmol) in dichloromethane (240 mL) was treated with powdered molecular sieves (16 g) followed by pyridinium dichromate (25.4 g). After stirring for 2 h, the mixture was diluted with ethyl acetate, then filtered through celite. Evaporation of the solvent afforded the crude product which was purified by chromatography on silica, eluting with 25-50% ethyl acetate-hexane gave the pure product (8 g, 72%).

¹H NMR CDCl₃ complex due to presence of rotamers (aldehyde protons at δ9.46 and 9.56)

### b) Preparation of (R)-2-Vinyl-pyrrolidine-1-carboxylic acid, t-butyl ester

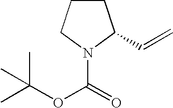

Sodium hydride (60% in oil, 1.65 g) was added to dry dimethylsulfoxide (50 mL) under argon, and the mixture was heated to 60° C. for 1 h. The solution was then cooled to 0° C. and a solution of methyltriphenylphosphonium bromide (14.4 g, 40.3 mmol) in dry dimethylsulfoxide (50 mL) added dropwise. After stirring for 30 min, a solution of (R)-2-formyl-pyrrolidine-1-carboxylic acid t-butyl ester (8.0 g, 40.2 mmol) in dry dimethylsulfoxide (15 mL) was added dropwise. The solution was stirred for 18 h at room temperature, then partitioned between water and 70% ethyl acetate-hexane. The organics were dried and evaporated to dryness. Chromatography on silica, eluting with 10% ethyl acetate-hexane gave the pure product (6.3 g, 79%)

¹H NMR CDCl₃ δ: 1.44 (9H, s), 1.6-2.1 (4H, m), 3.38 (2H, t), 4.26 (1H, b s), 5.04 (2H, m), 5.71 (1H, m).

### c) Preparation of (R)-2-(2-Hydroxy-ethyl)-pyrrolidine-1-carboxylic acid, t-butyl ester

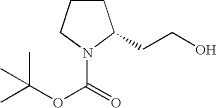

To a solution of (R)-2-vinyl-pyrrolidine-1-carboxylic acid t-butyl ester (6.3 g, 32 mmol) in dry tetrahydrofuran (130 mL) at 0° C. under argon was added 0.5M 9-BBN (192 mL). The solution was then refluxed for 18 h, then cooled. Ethanol (80 mL) was added cautiously followed by 2M sodium hydroxide (100 mL), followed by 30% hydrogen peroxide (60 mL) dropwise with cooling. The mixture was then refluxed for 3 h, then allowed to cool and diluted with brine. The mixture was extracted with ethyl acetate (×3), and the combined organics washed with brine, dried and evaporated to afford the crude product. Chromatography on silica, eluting with 20-40% ethyl acetate-hexane gave the pure product (6.7 g, 98%)

¹H NMR CDCl₃ δ: 1.47 (9H, s), 1.63 (2H, b s), 1.8-2.0 (4H, m), 3.16 (2H, m), 3.59 (2H, m), 4.13 (1H, b s), 4.37 (1H, b s)

### d) Preparation of (R)-2-Pyrrolidin-2-yl-ethanol hydrochloride

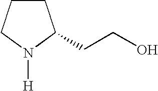

A solution of (R)-2-(2-hydroxy-ethyl)-pyrrolidine-1-carboxylic acid t-butyl ester (2.5 g, 11.6 mmol) in ethanol (10 mL) was treated with saturated ethanolic hydrogen chloride (30 mL). After stirring for 30 min, the solution was evaporated to dryness to afford the crude product (1.87 g, 100%) which was used directly in the next step.

### e) 2-[(R)-1-(4-Methyl-3-nitro-benzenesulfonyl)-pyrrolidin-2-yl]-ethanol

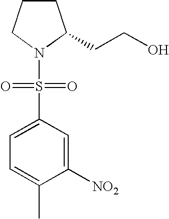

A mixture of (R)-2-pyrrolidin-2-yl-ethanol hydrochloride (5.1 g, 32.5 mmol) in dichloromethane (100 mL) was treated with diisopropylethylamine (15 mL) followed by 4-methyl-3-nitro-benzenesulfonyl chloride (8.4 g, 35.7 mmol) in dichloromethane (75 mL). The mixture was stirred at room temperature for 18 h under argon. The reaction mixture was treated with sodium bicarbonate solution then extracted with dichloromethane. The organic extracts were dried and evaporated to dryness. Chromatography on silica, eluting with 0-2.5% methanol in dichloromethane gave the pure product (10.3 g, -100%). Mass spectrum MH⁺ 315.

### f) (R-2-[2-(2-Methoxy-ethoxymethoxy)-ethyl]-1-(4-methyl-3-nitro-benzenesulfonyl)-pyrrolidine

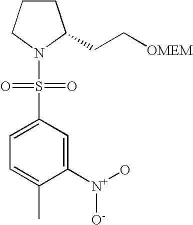

A solution of 2-[(R)-1-(4-methyl-3-nitro-benzenesulfonyl)pyrrolidin-2-yl]-ethanol (1.0 g, 3.18 mmol) In dichloromethane containing diisopropylethylamine (0.85 mL) was treated with MEM chloride (0.6 g, 4.8 mmol) in dichloromethane (2 mL) at room temperature under argon. After stirring for 18 h, the reaction mixture was poured onto water and extracted with dichloromethane (2×). The organic extracts were combined, dried and evaporated, then chromatographed over silica using 40-70% ethyl acetate/hexane as eluent to afford the title compound (1.13 g, 89%) Mass spectrum MH⁺ 403.

### g) 6-(R)-2-[2-(2-Methoxy-ethoxymethoxy)-ethyl]-pyrrolidine-1-sulfonyl)1H-indole

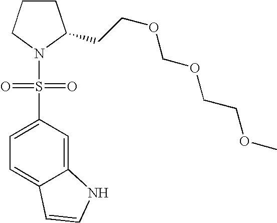

A solution of (R-2-[2-(2-methoxy-ethoxymethoxy)ethyl]-1-(4-methyl-3-nitro-benzenesulfonyl)-pyrrolidine (1.13 g, 2.8 mmol) in dimethylformamide dimethyl acetal (8 mL) containing dimethylformamide (1 mL) was heated to 100° C. for 8 h, then evaporated to dryness. The crude intermediate was then dissolved in tetrahydrofuran (15 mL) and methanol (15 mL), then Raney nickel (1 spoonful) added. Addition of hydrazine hydrate (2 mL) over a 2 h period was carried out, keeping the internal temperature at 45° C. The reaction mixture was then cooled, and filtered, then evaporated to dryness. Chromatography on silica using 40-70% ethyl acetate/hexane as eluent afforded the pure product (0.66 g, 62%). Mass spectrum MH⁺ 383.

### h) 2-[(R)-1-(1H-lndole-6-sulfonyl)-pyrrolidin-2-yl]-ethanol

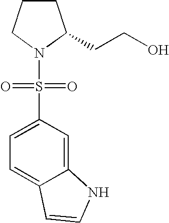

A solution of 6-(R)-2-[2-(2-methoxy-thoxymethoxy)ethyl]-pyrrolidine-1-sulfonyl}-1H-indole (0.6 g, 1.57 mmol) in dimethylformamide (10 mL) was treated with 60% sodium hydride (80 mg, 1.9 mmol) followed by benzenesulfonyl chloride (305 mg, 1.73 mmol). After stirring for 3 h, the reaction mixture was poured onto brine and extracted with ethyl acetate. The organic layers were combined, washed with brine, dried and evaporated to afford the protected indole-N-phenylsulfonamide. This intermediate was dissolved in dioxan (20 mL) and treated with 5M HCl (5 mL), then allowed to stir overnight at room temperature. The reaction mixture was poured onto brine and extracted with ethyl acetate. The organic layer was washed with brine, dried and evaporated to afford the deprotected alcohol. Mass spectrum MH⁺ 435. Removal of the indole-N-phenylsulfonamide protecting group was accomplished by treatment with 10% sodium hydroxide solution (3 mL) in dioxan (10 mL) at room temperature. After stirring for 18 h, the reaction mixture was poured onto water and extracted with ethyl acetate. The organic layer was washed with brine, dried and evaporated. Chromatography of the crude product on silica using 30-80% ethyl acetate/hexane as eluent afforded the pure product (270 mg, 65%). Mass spectrum MH⁺ 295.

### i) 6-[(R)-2-(2-Bromo-ethyl)-pyrrolidine-1-sulfonyl]-1H-indole (title compound)

A solution of 2-[(R)-1-(1H-indole-6-sulfonyl)-pyrrolidin-2-yl]-ethanol (190 mg, 0.64 mmol) in dichloromethane (4 mL) was cooled to 0° C. and then treated with carbon tetrabromide (254 mg, 0.77 mmol) followed by triphenylphosphine (200 mg, 0.77 mmol). Chromatography of the reaction mixture directly onto a short column of silica, and elution with dichloromethane afforded the pure product (200mg, 88%). Mass spectrum MH⁺ 358.

### DESCRIPTION 5

### 6-[(R)-2-(2-Bromo-ethyl)-pyrrolidine-1-sulfonyl]-1H-indazole (D2)

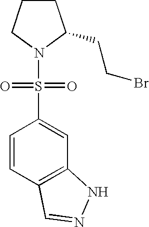

The title compound was prepared from Description 1e using the following steps:—

### a) 2-[(R)-1-(3-Amino-4-methyl-benzenesulfonyl)-pyrrolidin-2-yl]-ethanol

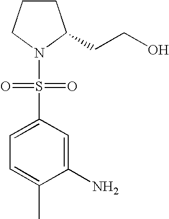

To a solution of 2-[(R)-1-(4-methyl-3-nitro-benzenesulfonyl)-pyrrolidin-2-yl]-ethanol (8.2 g, 26 mmol) in tetrahydrofuran (80 mL) and methanol (80 mL) was added Raney nickel (1 spoonful). The mixture was then treated with hydrazine hydrate (3×4 mL additions over a 2 h period) keeping the internal temperature at 45° C. After cooling the catalyst was filtered off, and the solution evaporated to dryness to afford the pure product (6.97 g, 95%). Mass spectrum MH⁺ 285.

### b) 2-[(R)-1-(1H-Indazole-6-sulfonyl)-pyrrolidin-2-yl]-ethanol

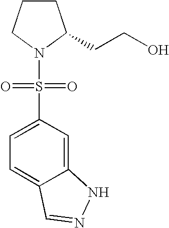

A solution of 2-[(R)-1-(3-amino-4-methyl-benzenesulfonyl)-pyrrolidin-2-yl]-ethanol (3.4 g, 11.9 mmol) in acetic acid (40 mL) and water (10 mL) was cooled in an ice bath, then a solution of sodium nitrite (1.24 g, 18 mmol) in water (3 mL) was added dropwise. The mixture was allowed to warm to room temperature then stirred for 18 h. The reaction mixture was then neutralised with 40% sodium hydroxide (˜65 mL) with cooling, and extracted with ethyl acetate (×2). The combined organic layer was washed with brine, dried and evaporated to afford the crude product. Chromatography on silica with 50-80% ethyl acetate in hexane as eluent afforded the pure product (2.5 g, 71%).Mass spectrum MH⁺ 296.

### c) 6-[(R-2-(2-Bromo-ethyl)pyrrolidine-1-sulfonyl]-1H-indazole (title compound)

6-[(R)-2-(2-Bromo-ethyl)-pyrrolidine-1-sulfonyl]-1H-indazole was prepared in 77% yield from 2-[(R)-1-(1H-indazole-6-sulfonyl)-pyrrolidin-2-yl]-ethanol using a procedure similar to that in Description 1i. Mass spectrum MH⁺ 359.

### DESCRIPTION 6

### 6-[(R)-2-(2-Bromo-ethyl)-pyrrolidine-1-sulfonyl]-4H-benzo[1,4]oxazin-3-one (D3)

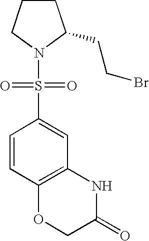

The title compound was prepared using the following steps:—

### a) 6-[(R-2-(2-Hydroxy-ethyl)-pyrrolidine-1-sulfonyl]4H-benzo[1,4]oxazin-3-one

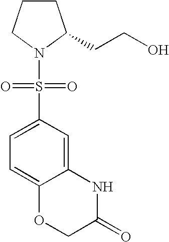

A mixture of (R)-2-pyrrolidin-2-yl-ethanol hydrochloride (3.3 g, 18.6 mmol) in dichloromethane (60 mL) was treated with diisopropylethylamine (9 mL) followed by 3oxo-3,4-dihydro-2H-benzo[1,4]oxazine-6-sulfonyl chloride (C R Acad Science Ser C. 1970, 270, 1601) (5.2 g, 21 mmol) in dichloromethane (20 mL). The mixture was stirred at room temperature for 18 h under argon. The reaction mixture was treated with sodium bicarbonate solution then extracted with dichloromethane. The organic extracts were dried and evaporated to dryness. Chromatography on silica, eluting with 2-5% methanol in dichloromethane gave the pure product (6.0 g, 98%). Mass spectrum MH⁺ 327.

### b) 6-[(R)-2-(2-Bromo-ethyl)-pyrrolidine-1-sulfonylj4H-benzo[1,4]oxazin-3-one (title compound)

6-[(R-2-2-Bromo-ethyl)-pyrrolidine-1-sulfonyl]-4H-benzo[1,4]oxazin-3-one was prepared in 86% yield from 6-[(R)-2-(2-hydroxy-ethyl)-pyrrolidine-1-sulfonyll-4H-benzo[1,4]oxazin-3-one using a procedure similar to that in Description 1i. Mass spectrum MH⁺ 390.

### EXAMPLE 1

### 6-(2-(2-[4-(1H-Indol-3-yl)-piperidin-1-yl]-ethyl}-pyrrolidine-1-sulfonyl)-1H-indole (E1)

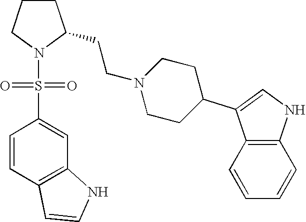

A solution of 6-[(R)-2-(2-bromo-ethyl)pyrrolidine-1-sulfonyl]-1H-indole (Description 1; 160 mg, 0.44 mmol) and 3-piperidin-4-yl-1H-indole hydrochloride (114 mg, 0.48 mmol) in dimethylformamide (5 mL) containing sodium bicarbonate (350 mg) and sodium iodide (80 mg) was heated to 100° C. for 20 h. The reaction mixture was the poured onto water, pH adjusted to ˜8, and extracted with ethyl acetate. The organic extracts were washed with brine dried and evaporated to dryness. The crude product was chromatographed on silica using 2-3.5% methanol in dichloromethane containing 0.5% aqueous ammonia to afford the title compound (190 mg, 90%). Mass spectrum MH⁺ 477.

¹H NMR CDCl₃ δ: 1.4-1.9 (7H, m), 2.0-2.25 (5H, m), 2.37-2.6 (2H, m), 2.88 (1H, b t), 3.03 (1H, b d), 3.14 (1H, b d), 3.28 (1H, m), 3.55 (1H, m), 3.80 (1H, m), 6.63 (1H, b s), 7.01 (1H, d), 7.08-7.24 (2H, m), 7.38 (2H, m), 7.58 (1H, d), 7.67 (1H, d), 7.75 (1H, d), 7.96 (2H, m), 8.57 (1H, b s).

### Examples E2- E17 were prepared in an analogous manner to E1, using the appropriate bromide D1, D2 or D3 and the appropriate amine as indicated in Table 1.

TABLE 1

| Example | amine | Product | MH⁺ |
|---|---|---|---|
| E2 | 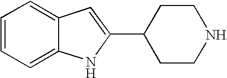 | 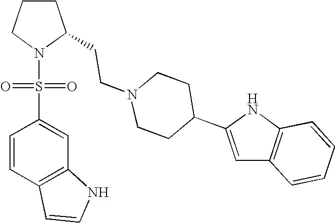 | 477 |
| E3 | 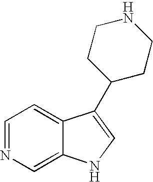 | 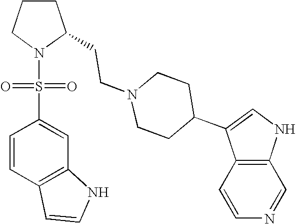 | 478 |
| E4 | 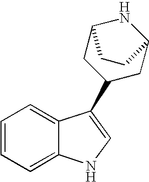 | 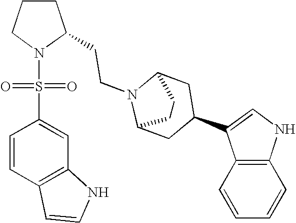 | 503 |
| E5 | 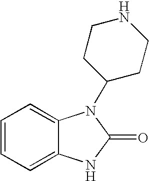 | 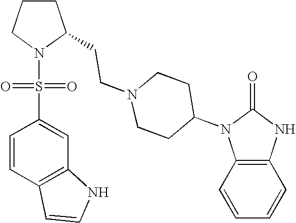 | 494 |
| E6 | 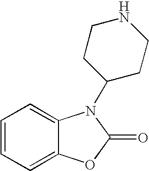 | 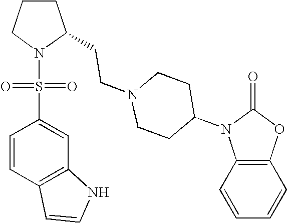 | 495 |
| E7 | 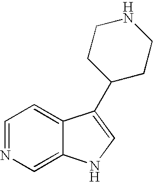 | 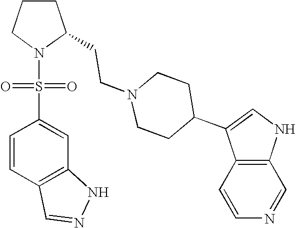 | 479 |
| E8 | 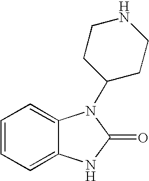 | 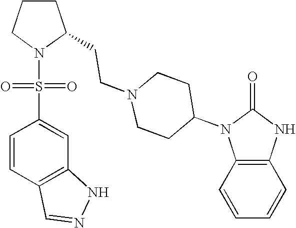 | 495 |
| E9 | 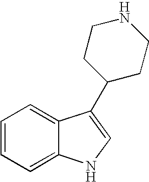 | 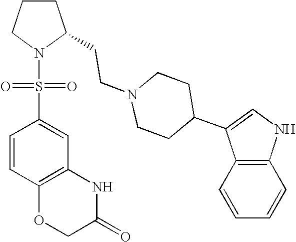 | 509 |
| E10 | 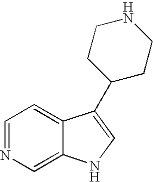 | 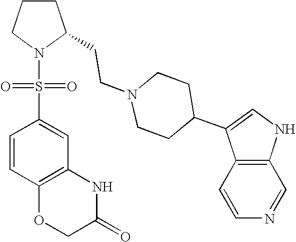 | 510 |
| E11 | 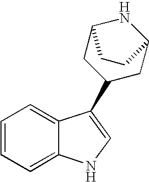 | 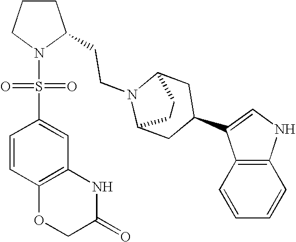 | 535 |
| E12 | 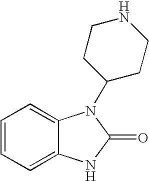 | 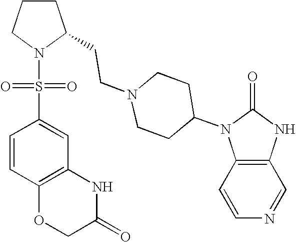 | 526 |
| E13 | 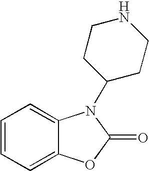 | 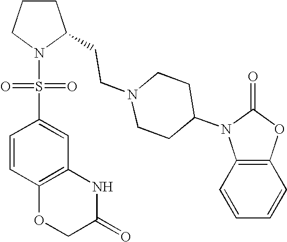 | 527 |
| E14 | 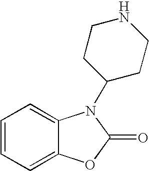 | 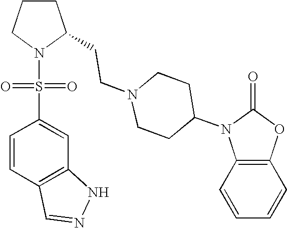 | 496 |
| E15 | 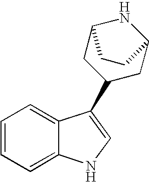 | 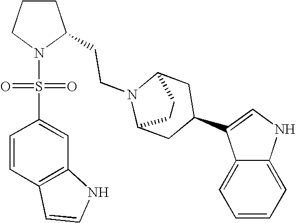 | 504 |
| E16 | 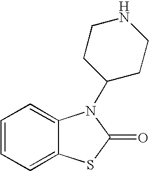 | 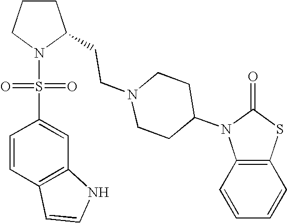 | 511 |
| E17 | 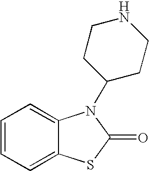 | 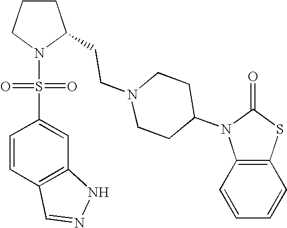 | 512 |

### EXAMPLE 18

### 6-((R)-2-{2-[4-(4-Chloro-phenoxy)-piperidin-1-yl]-ethyl}-pyrrolidine-1-sulfonyl)-1H-indole (E18)

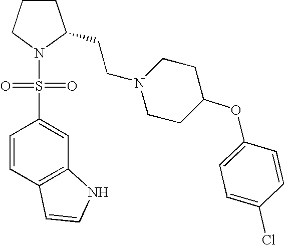

A solution of 6-[(R)-2-(2-bromo-ethyl)-pyrrolidine-1-sulfonyl)-1H-indole (Description 1; 140 mg, 0.39 mmol) and 4-(4-chloro-phenoxy)-piperidine hydrochloride (110 mg, 0.44 mmol) in dimethylformamide (5 mL) containing sodium bicarbonate (350 mg) and sodium iodide (80 mg) was heated to 100° C. for 20 h. The reaction mixture was the poured onto water, pH adjusted to ˜8, and extracted with ethyl acetate. The organic extracts were washed with brine dried and evaporated to dryness. The crude product was chromatographed on silica using 2-3.5% methanol in dichloromethane containing 0.5% aqueous ammonia to afford the title compound (130 mg, 68%). Mass spectrum MH⁺ 488.

¹H NMR CDCl₃ δ: 1.4-1.95 (8H, m), 1.96-2.14 (2H, m), 2.17-2.39 (2H, m), 2.4-2.55 (2H, m), 2.7-2.95 (2H, m), 3.25 (1H, m), 3.42 (1H, m), 3.80 (1H, m), 4.29 (1H, m), 6.63 (1H, m), 6.83 (2H, m), 7.20 (2H, m), 7.42 (1H, t), 7.55 (1H, d d), 7.75 (1H, d), 7.97 (1, s) 8.69 (1H, b s).

Examples E19- E29 were prepared in an analogous manner to E18, using the appropriate bromide D4, D5 or D6 and the appropriate amine as indicated in Table 2. NMR and mass spectral data were consistent with the structures shown.

TABLE 2

| Example | amine | Product | MH⁺ |
|---|---|---|---|
| E19 | 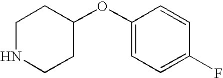 | 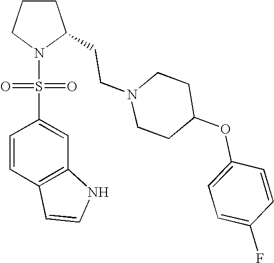 | 472 |
| E20 | 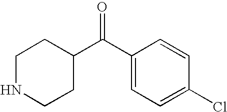 | 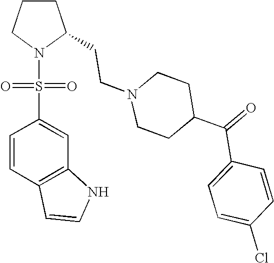 | 501 |
| E21 | 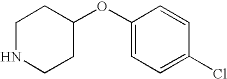 | 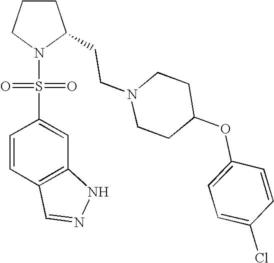 | 490 |
| E22 | 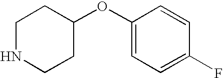 | 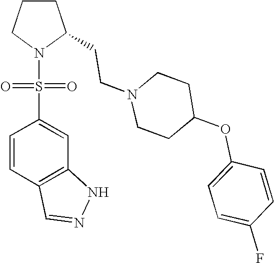 | 473 |
| E23 | 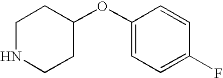 | 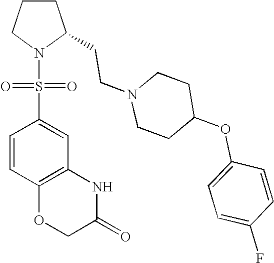 | 504 |
| E24 | 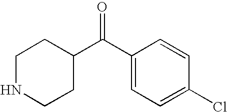 | 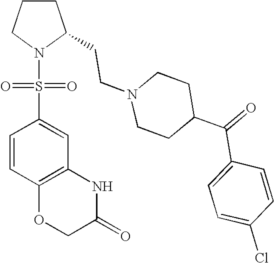 | 533 |
| E25 | 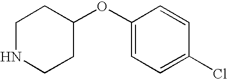 | 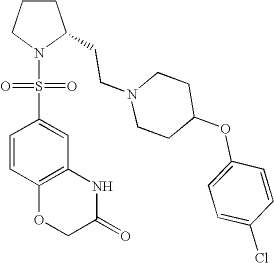 | 521 |
| E26 | 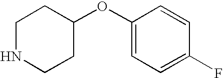 | 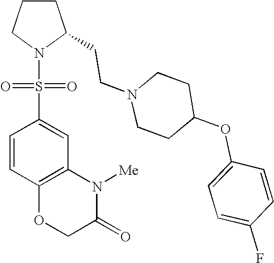 | 518 |
| E27 | 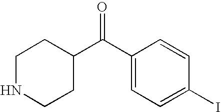 | 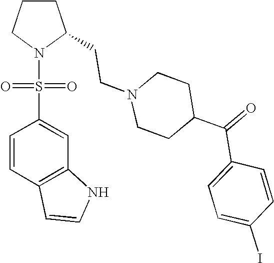 | 592 |
| E28 | 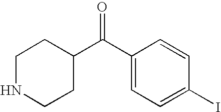 | 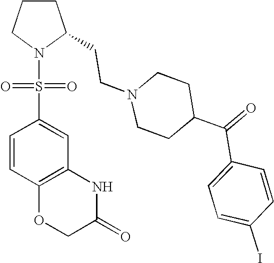 | 624 |
| E29 | 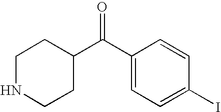 | 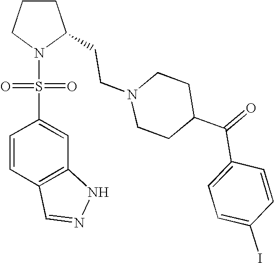 | 593 |

### Pharmacological Data

### [³H]-5-Carboxamidotryptamine Binding to Human 5HT₇ Receptor Clones Expressed in HEK 293 Cells In Vitro

The affinity of the compounds of this invention for the 5-HT₇ receptor binding site can be determined by methods described in WO 97/29097. Briefly, affinity is determined by assessing a compound's ability to displace [³H]-5-carboxamidotryptamine from 5HT₇ receptor clones expressed in 293 cells (To, Z. P., et al (1995) Br. J. Pharmacol., 15, 107; Sleight, A. J., et al (1995) Mol. Pharmacol., 47, 99). All Example compounds were tested and were found to have a pKi in the range 8.3-9.3.

What is claimed is:

1. A compound of formula (I) or a pharmaceutically acceptable salt thereof: 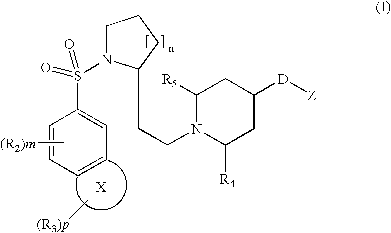
in which
X is a 5 or 6 membered heterocyclic ring containing 1 to 3 heteroatoms selected from oxygen, nitrogen and sulfur;
R₂ is halogen, C₁₋₆alkyl, haloC₁₋₆alkyl, hydroxy, C₁₋₆alkoxy or C₁₋₆alkylthio;
R₃ is hydrogen, C₁₋₆alkyl, hydroxy or oxo;
m is 0, 1 or 2;
p is 0, 1 or 2;
n is 1 or 2;
R₄ and R₅ are both hydrogen or R₄ and R₅ combine together to form a further group
—(CH2)q- where q is 2 or 3;
D is a single bond, C₁₋₄alkylene, C═O or oxygen;
Z is either
a group (a): 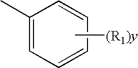
in which R₁ is halogen, C₁₋₆alkyl, cyano, haloC₁₋₆alkyl, C₃₋₇cycloalkyl, C₁₋₆alkoxy, hydroxy, amino, mono- or di-C₁₋₆alkylamino, acylamino, nitro, carboxy, C₁₋₆alkoxycarbonyl, C₁₋₆alkylthio, C₁₋₆alkylsulfinyl, C₁₋₆alkylsulfonyl, sulfamoyl, mono- and di-C₁₋₆alkylsulfamoyl, carbamoyl, mono- or di-C₁₋₆alkylcarbamoyl, C₁₋₆alkylsulfonamido, arylsulfonamido, aryl, arylC₁₋₆alkyl, arylC₁₋₆alkoxy, aryloxy or arylthio, and y is 0, 1, 2 or 3;
or a group (b): 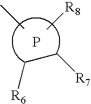
in which P is a 5 membered heterocyclic ring containing 1 to 3 heteroatoms selected from oxygen, nitrogen and sulfur; R₈ is hydrogen, C₁₋₆alkyl, hydroxy or oxo; R₆ and R₇ form the residue of a phenyl ring or a 6 membered heteroaryl ring comprising from one to three heteroatoms selected from nitrogen, oxygen and sulfur and optionally substituted with one or two substitutents which may be the same or different and selected from the group consisting of halogen, C₁₋₆alkyl, cyano, haloC₁₋₆alkyl, C₃₋₇cycloalkyl, C₁₋₆alkoxy, hydroxy, amino, mono- or di-C₁₋₆alkylamino, acylamino, nitro, carboxy, C₁₋₆alkoxycarbonyl, C₁₋₆alkylthio, C₁₋₆alkylsulfinyl, C₁₋₆alkylsulfonyl, sulfamoyl, mono- and di-C₁₋₆alkylsulfamoyl, carbamoyl, mono- and di-C₁₋₆alkylcarbamoyl, C₁₋₆alkylsulfonamido, arylsulfonamido, aryl, arylC₁₋₆alkyl, arylC₁₋₆alkoxy, aryloxy and arylthio.

2. A compound as claimed in claim 1, wherein X contains at least one nitrogen.

3. A compound as claimed in claim 2, wherein X contains a free NH group with a meta relationship with respect to the sulfonamide linkage.

4. A compound as claimed in claim 3, wherein X, together with the benzene ring to which it is fused, forms an indole, indoline, indazole, benzotriazole, benzimidazole or a benzoxazine ring.

5. A compound according to claim 1, wherein R₂ is C₁₋₆alkyl (particularly methyl), halogen (particularly fluoro or chloro), C₁₋₆alkoxy (particularly methoxy) or CF₃.

6. A compound according to claim 1, wherein m is 0.

7. A compound according to claim 1, wherein n is 1 and the stereochemistry of the pyrrolidine ring is R.

8. A compound according to claim 1, wherein R₄ and R₅ are both hydrogen.

9. A compound according to claim 1, having the general formula (Ia): 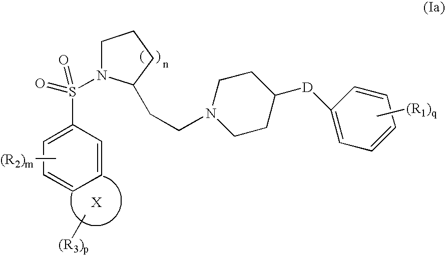
and D is C═O or oxygen;
or the general formula (Ib): 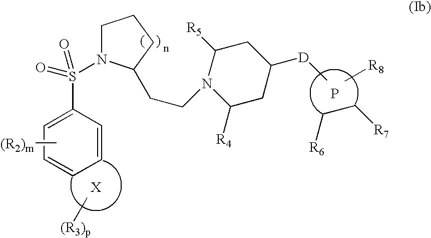
and D is a single bond or a C₁₋₄alkylene group.

10. A compound of formula (Ib) as claimed in claim 9, wherein P is pyrrolinyl, thienyl, furyl, imidazolyl, oxazolyl or thiazolyl.

11. A compound of formula (Ib) as claimed in claim 9, wherein R₆ and R₇ form a phenyl ring or a pyridyl ring.

12. A compound of formula (Ib) as claimed in claim 9, wherein D is a single bond.

13. A compound as claimed in claim 1, which is any of E1-E29 or a pharmaceutically acceptable salt thereof.

14. A process for the preparation of a compound of formula (I) as defined in claim 1 or a pharmaceutically acceptable salt thereof, which process comprises either:
(a) coupling a compound of formula (II): 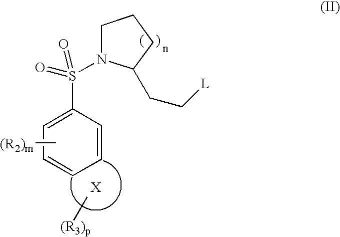
and L is a leaving group, e.g. halogen, with a compound of formula (III): 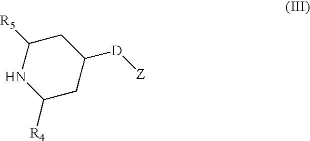
or
(b) formation from a compound of formula (IV) 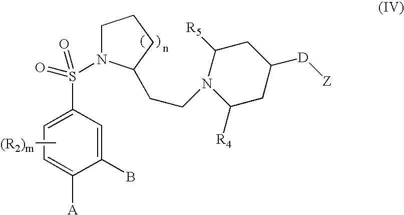
and A and B are appropriate functional groups for the formation of the ring X;
and optionally thereafter if appropriate for either (a) or (b):
converting a compound of formula (I) into a further compound of formula (I)
removing any protecting groups;
forming a pharmaceutically acceptable salt.

15. A pharmaceutical composition which comprises a compound as defined in claim 1 and a pharmaceutically acceptable carrier or excipient.

16. A process for preparing a pharmaceutical composition as defined in claim 15, which comprises admixing a compound as defined in claim 1 and a pharmaceutically acceptable carrier or excipient.

17. A compound or a composition as defined in claim 1 for use in therapy.

18. A compound or a composition as defined in claim 1 for use in the treatment of CNS and other disorders.

19. A compound or a composition as defined in claim 1 for use in the treatment of depression, migraine, anxiety, stroke, pain and/or sleep disorders.

20. The use of a compound or a composition as defined in claim 1 in the manufacture of a medicament for the treatment of CNS and other disorders.

21. The use as claimed in claim 20, wherein the medicament is for the treatment of depression, migraine, anxiety, stroke, pain and/or sleep disorders.

22. A method of treatment of CNS and other disorders, in mammals including humans, which comprises administering to the sufferer a therapeutically effective amount of a compound or a composition as defined in claim 1.
